(19) United States
(12) Patent Application Publication
(10) Pub. No.: US 2021/0361626 A1
(43) Pub. Date: Nov. 25, 2021

(54) CRYSTALLINE PRODUCT

(71) Applicant: Johnson Matthey Public Limited Company, London (GB)

(72) Inventors: Amanda BUIST, Cambridge (GB); Thierry BONNAUD, Cambridge (GB); Richard EDWARDS, Cambridge (GB); Adam PATTERSON, Cambridge (GB); Mark WRIGHT, Cambridge (GB)

(21) Appl. No.: 17/444,573

(22) Filed: Aug. 6, 2021

Related U.S. Application Data
(63) Continuation of application No. PCT/GB2020/050301, filed on Feb. 11, 2020.

(30) Foreign Application Priority Data
Feb. 11, 2019 (GB) 1901841.5

(51) Int. Cl. A61K 31/4184; A61K 47/20; A61K 47/12

(52) U.S. Cl. CPC A61K 31/4184; A61K 47/12; A61K 47/20

(57) ABSTRACT

The present invention provides a molecular complex of binimetinib, which is binimetinib dimethylsulfoxide (DMSO) solvate. It is also an object of the present invention to provide a molecular complex of binimetinib which is a crystalline molecular complex of binimetinib and citric acid. The present invention also relates to methods for the preparation of these molecular complexes.

### CROSS-REFERENCE TO RELATED APPLICATIONS

This application is a continuation of International Application No. PCT/GB2020/050301, filed Feb. 11, 2020, which claims priority to GB application No. 1901841.5, filed Feb. 11, 2019, the disclosures of which are incorporated herein by reference in their entireties for all purposes.

### FIELD OF THE DISCLOSURE

The present invention relates to molecular complexes of binimetinib, and methods for the preparation of the molecular complexes. The invention also relates to the molecular complexes for use in the inhibition of MEK activity, or the treatment of a hyperproliferative disorder.

### BACKGROUND OF THE DISCLOSURE

Binimetinib has the IUPAC name of 5-[(4-bromo-2-fluorophenyl)amino]-4-fluoro-N-(2-hydroxyethoxy)-1-methyl-1H-1,3-benzodiazole-6-carboximide and has the chemical structure shown below:

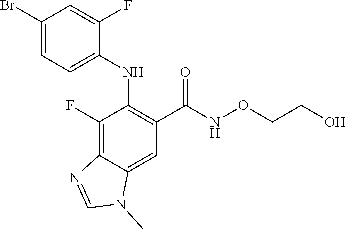 [SMILES: Cn1cnc2c(F)c(Nc3ccc(Br)cc3F)c(C(=O)NOCCO)cc21]

WO2016/131406 (to Crystal Pharmatech Co., Ltd) describes binimetinib Forms A and B. Form A is anhydrous crystalline polymorph of binimetinib.

The compound binimetinib may exist in a number of polymorphic forms and many of these forms may be undesirable for producing pharmaceutically acceptable compositions. This may be for a variety of reasons including lack of stability, high hygroscopicity, low aqueous solubility and difficulty in handing.

### Definitions

The term “about” or “approximately” means an acceptable error for a particular value as determined by a person of ordinary skill in the art, which depends in part on how the value is measured or determined.

In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range. In certain embodiments and with reference to X-ray powder diffraction two-theta peaks, the terms “about” or “approximately” means within ±0.2°2θ.

The term “ambient temperature” means one or more room temperatures between about 15° C. to about 30° C., such as about 15° C. to about 25° C.

The term “anti-solvent” refers to a first solvent which is added to a second solvent to reduce the solubility of a compound in that second solvent. The solubility may be reduced sufficiently such that precipitation of the compound from the first and second solvent combination occurs.

The term “consisting” is closed and excludes additional, unrecited elements or method steps in the claimed invention.

The term “consisting essentially of” is semi-closed and occupies a middle ground between “consisting” and “comprising”. “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to”. The term “comprising” encompasses three alternatives, namely (i) “comprising”, (ii) “consisting”, and (iii) “consisting essentially of”.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

The term “molecular complex” is used to denote a crystalline material composed of two or more different components which has a defined single-phase crystal structure. The components are held together by non-covalent bonding, such as hydrogen bonding, ionic bonding, van der Waals interactions, π-π interactions, etc. The term “molecular complex” includes solvates, salts, co-crystals and salt/co-crystal hybrids. In one embodiment, the molecular complex is a solvate. In one embodiment, the molecular complex is a salt. In another embodiment, the molecular complex is a co-crystal. In another embodiment, the molecular complex is a salt/co-crystal hybrid.

Without wishing to be bound by theory, it is believed that when the molecular complex is a co-crystal, the co-crystal demonstrates improved properties, such as crystallisation and bioavailability properties.

The molecular complexes may be distinguished from mixtures of binimetinib and the selected molecular complex former, such as citric acid, by standard analytical means which are well known to those skilled in the art, for example X-ray powder diffraction (XRPD), single crystal X-ray diffraction, or differential scanning calorimetry (DSC). The molar ratio of the components of the molecular complex may be determined using, for example, HPLC or ¹H-NMR.

The terms “polymorph,” “polymorphic form” or related term herein, refer to a crystal form of one or more molecules of binimetinib, or binimetinib molecular complex thereof that can exist in two or more forms, as a result different arrangements or conformations of the molecule(s) in the crystal lattice of the polymorph.

The term “pharmaceutical composition” is intended to encompass a pharmaceutically effective amount of binimetinib of the invention and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

The term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

The term “patient” refers to an animal, preferably a patient, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/prevented, but has been deemed by a physician, clinician or other medical professional to be at risk for developing said disorder, disease or condition.

The term “solvate” refers to a combination or aggregate formed by one or more molecules of a solute e.g. binimetinib, and one or more molecules of a solvent. The one or more molecules of the solvent may be present in stoichiometric or non-stoichiometric amounts to the one or more molecules of the solute.

The terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a molecular complex provided herein, with or without other additional active agents, after the onset of symptoms of a disease.

The term “overnight” refers to the period of time between the end of one working day to the subsequent working day in which a time frame of about 12 to about 18 hours has elapsed between the end of one procedural step and the instigation of the following step in a procedure.

### BRIEF DESCRIPTION OF THE FIGURES

Certain aspects of the embodiments described herein may be more clearly understood by reference to the drawings, which are intended to illustrate but not limit, the invention, and wherein:

FIG. 1 shows a representative X-ray powder diffraction (XRPD) pattern for the binimetinib DMSO solvate described in Example 6.

FIG. 2 shows a view of binimetinib DMSO solvate from the single crystal structure, showing the atom numbering scheme. Anisotropic atomic displacement ellipsoids for the non-hydrogen atoms are shown at the 50% probability level. Hydrogen atoms are displayed with an arbitrarily small radius.

FIG. 3 shows a representative TGA thermogram and a DSC thermogram of binimetinib DMSO solvate.

FIG. 4 shows a representative ¹H-NMR spectrum of binimetinib DMSO solvate.

FIG. 5 shows a representative X-ray powder diffraction (XRPD) pattern for the binimetinib citric acid molecular complex described in Example 11.

FIG. 6 shows a representative TGA thermogram and a DSC thermogram of binimetinib citric acid molecular complex.

FIG. 7 shows a representative ¹H-NMR spectrum of binimetinib citric acid molecular complex.

FIG. 8 shows a representative XRPD overlay of binimetinib citric acid before storage (bottom), binimetinib citric acid after storage at 40° C./75% RH (relative humidity) for 10 days (middle) and binimetinib citric acid after storage at 25° C./97% RH after 10 days (top).

FIG. 9 shows a representative FT-IR overlay of (a) binimetinib citric acid molecular complex, (b) binimetinib free base, and (c) citric acid anhydrate.

FIG. 10 shows a representative Raman overlay of (a) binimetinib citric acid molecular complex, (b) binimetinib free base, and (c) citric acid anhydrate.

FIGS. 11A-C illustrate how centrifugal forces are applied to particles in the Speedmixer™. FIG. 11A is a view from above showing the base plate and basket. The base plate rotates in a clockwise direction.

FIG. 11B is a side view of the base plate and basket.

FIG. 11C is a view from above along line A in FIG. 11B. The basket rotates in an anti-clockwise direction.

FIG. 12 is a representative photograph depicting a Rondol Microlab 10 mm hot melt extruder.

FIG. 13 is a representative photograph depicting the hot melt extruder screw design with conveying and mixing elements.

FIG. 14 is a representative photograph depicting the solvent addition set up for the hot melt extruder.

### DESCRIPTION OF THE INVENTION

The present invention seeks to overcome the disadvantages associates with the prior art. The invention provides a molecular complex of binimetinib, which is binimetinib dimethylsulfoxide (DMSO) solvate. In certain embodiments, the molecular complex is purifiable. In certain embodiments and depending on time, temperature and humidity, the molecular complex is stable. In certain embodiments, the molecular complex is easy to isolate and handle. In certain embodiments, the process for preparing the molecular complex is scalable.

It is also an object of the present invention to provide a molecular complex of binimetinib which is a crystalline molecular complex of binimetinib and citric acid. In certain embodiments, the crystalline molecular complex is purifiable. In certain embodiments, the crystalline molecular complex is stable. In certain embodiments, the crystalline molecular complex is easy to isolate and handle. In certain embodiments, the process for preparing the crystalline molecular complex is scalable.

The crystalline forms described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The purity of the crystalline forms provided herein may be determined by standard analytical methods, such as thin layer chromatography (TLC), gas chromatography, high performance liquid chromatography (HPLC), and mass spectrometry (MS).

Binimetinib DMSO Solvate

In one aspect, the present invention provides a molecular complex of binimetinib which is crystalline binimetinib DMSO solvate. The solvate consists of one molecule of binimetinib to one molecule of DMSO.

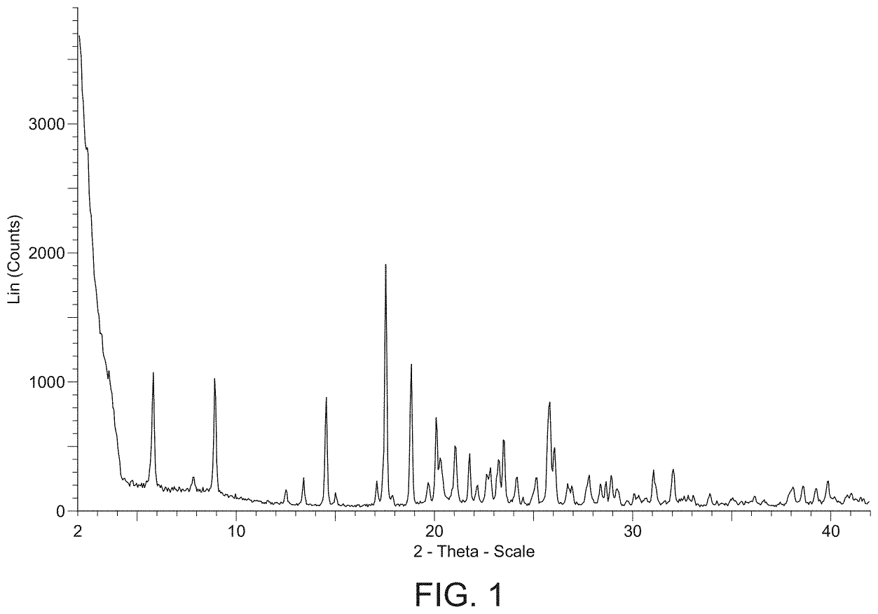

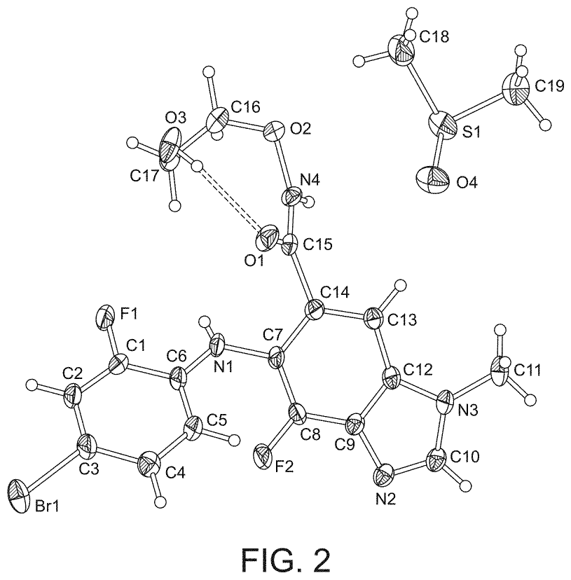

The solvate may have an X-ray powder diffraction pattern comprising one or more peaks (for example 1, 2, 3, 4, 5, 6, 7, or 8 peaks) selected from the group consisting of about 5.8, 7.9, 8.9, 12.5, 13.4, 14.5, 15.1, 17.1, 17.6, 17.9, 18.8, 19.7, 20.1, 20.3, 21.0, 21.8, 22.2, 22.7, 22.8, 23.3, 23.5, 24.2, 24.5, 25.2, 25.8, 26.1, 26.8, 27.0, 27.7, 27.8, 28.4, 28.7, 29.0, 29.2, 29.8, 30.1, 30.3, and 30.7 degrees two-theta ±0.2 degrees two-theta. In one embodiment, the solvate may have an X-ray powder diffraction pattern comprising peaks at about 5.8, 8.9, 14.5, 17.6, 18.8, 20.1, 23.5, and 25.8 degrees two-theta ±0.2 degrees two-theta. In one embodiment, the solvate may have the X-ray powder diffraction pattern substantially as shown in FIG. 1. The asymmetric unit of the solvate appears to contain one fully ordered molecule of binimetinib and one fully ordered molecule of DMSO (see FIG. 2).

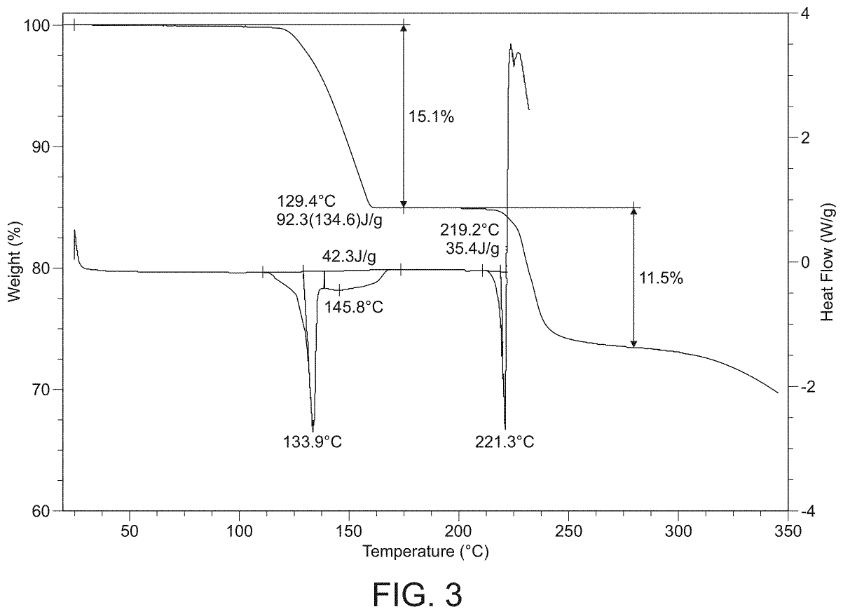

The solvate may have a DSC thermogram comprising an endothermal event with an onset temperature of about 129.4° C.; and another endothermal event with an onset temperature of about 219.2° C. The solvate may have a DSC thermogram comprising an endothermal event with a peak at about 133.9° C.; and another endothermal event with a peak at about 221.3° C. In one embodiment, the solvate may have a DSC thermogram substantially as shown in FIG. 3.

The solvate may have a TGA thermogram comprising a first mass loss of about 15.1% when heated from about 100° C. to about 175° C.; and a second mass loss of about 11.5% when heated from about 175° C. to about 280° C. In one embodiment, the solvate may have a TGA plot substantially as shown in FIG. 3.

Binimetinib DMSO solvate may be prepared by a process comprising the steps of:
(a) contacting binimetinib with DMSO; and
(b) forming a solution of binimetinib in DMSO.

The quantity of DMSO is not particularly limiting provided there is enough DMSO to substantially dissolve the binimetinib to form a solution. If a suspension remains on contacting the binimetinib with DMSO, a second quantity or further quantities of DMSO may be added until a solution is formed. The ratio of binimetinib to DMSO solvent may be in the range of about 1 g of binimetinib:about 0.5 ml to about 25 ml of DMSO, for example, about 1 g of binimetinib:about 1.5 ml to about 20 ml of DMSO.

The binimetinib may be contacted with DMSO at ambient temperature or less. Alternatively, the binimetinib may be contacted with DMSO at a temperature greater than ambient i.e. greater than 30° C. and below the boiling point of the reaction mixture. The boiling point of the reaction mixture may vary depending on the pressure under which the contacting step is conducted. DMSO has a boiling point of 189° C. at atmospheric pressure (i.e. 1.0135×10⁵ Pa). In one embodiment, the contacting step may be carried out at one or more temperatures in the range of about 30° C. to about <189° C. In some embodiments, the contacting step is carried out at one or more temperatures ≥40° C. In some embodiments, the contacting step is carried out at one or more temperatures ≥50° C. In some embodiments, the contacting step is carried out at one or more temperatures ≥60° C. In some embodiments, the contacting step is carried out at one or more temperatures ≤150° C. In some embodiments, the contacting step is carried out at one or more temperatures ≤125° C. In some embodiments, the contacting step is carried out at one or more temperatures ≤115° C. In some embodiments, the contacting step is carried out at one or more temperatures ≤110° C. In some embodiments, the contacting step is carried out at one or more temperatures ≤105° C. In some embodiments, the contacting step is carried out at one or more temperatures ≤100° C. In one embodiment, the contacting step is carried out at one or more temperatures in the range of ≥70° C. to ≤100° C.

The dissolution of binimetinib may be encouraged through the use of an aid such as stirring, shaking and/or sonication.

The process may further comprise the step of recovering binimetinib DMSO solvate as a crystalline solid. The recovery of the crystalline DMSO solvate may comprise:
(c) treating the solution obtained in step (b) with an anti-solvent selected from the group consisting of water, an alcohol and a mixture thereof; and
(d) recovering the binimetinib DMSO solvate as a crystalline solid.

Any suitable anti-solvent which is miscible with DMSO may be used. The anti-solvent may be selected from the group consisting of water, methanol, ethanol, propanol (n- or i-), butanol (n-, i- or t-), a pentanol isomer, cyclopentanol, a hexanol isomer, cyclohexanol or mixtures thereof. In one embodiment, the anti-solvent is water. In another embodiment, the anti-solvent is isopropanol.

Sufficient anti-solvent is added until precipitation of binimetinib DMSO solvate occurs.

After the addition of the anti-solvent, the reaction mixture may be stirred or shaken at ambient temperature for a period of time until a slurry or suspension is formed e.g. overnight.

The recovery of the DMSO solvate may comprise evaporating the DMSO solvent under ambient temperature.

Alternatively, the reaction mixture of step (b) may be optionally filtered (e.g. polish filtered), heated to about 70° C. for about 5 minutes, cooled to ambient temperature over a period of time (e.g. less than 1 hour), before being treated with anti-solvent at ambient temperature or lower (for example, with certain mixtures of DMSO and anti-solvent).

Alternatively, the reaction mixture of step (c) may be stirred for a period of time (e.g. about 80 minutes) and then cooled to about 5° C. at about 1° C./minute. The reaction mixture may be stirred at about 5° C. for about 36 hours.

Howsoever the crystalline DMSO solvate is recovered, the separated solvate may be washed with alcohol and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the DMSO solvate desolvates and so when the solvate is known to desolvate within the temperature or pressure ranges given above, the drying conditions should be maintained below the desolvation temperature or vacuum.

In another aspect, the present invention relates to a pharmaceutical composition comprising binimetinib DMSO solvate as described herein and a pharmaceutically acceptable excipient.

In another aspect, the present invention relates to a method for inhibiting MEK activity in a patient comprising administering a therapeutically effective amount of binimetinib DMSO solvate as described herein to the patient.

In another aspect, the present invention relates to a method for the treatment of a hyperproliferative disorder in a patient comprising administering a therapeutically effective amount of binimetinib DMSO solvate to the patient.

In another aspect, the present invention relates to binimetinib DMSO solvate as described herein for use in inhibiting MEK activity.

In another aspect, the present invention relates to binimetinib DMSO solvate as described herein for use in the treatment of a hyperproliferative disorder.

Binimetinib Citric Acid Molecular Complex

In another aspect, the present invention provides a crystalline molecular complex of binimetinib and citric acid.

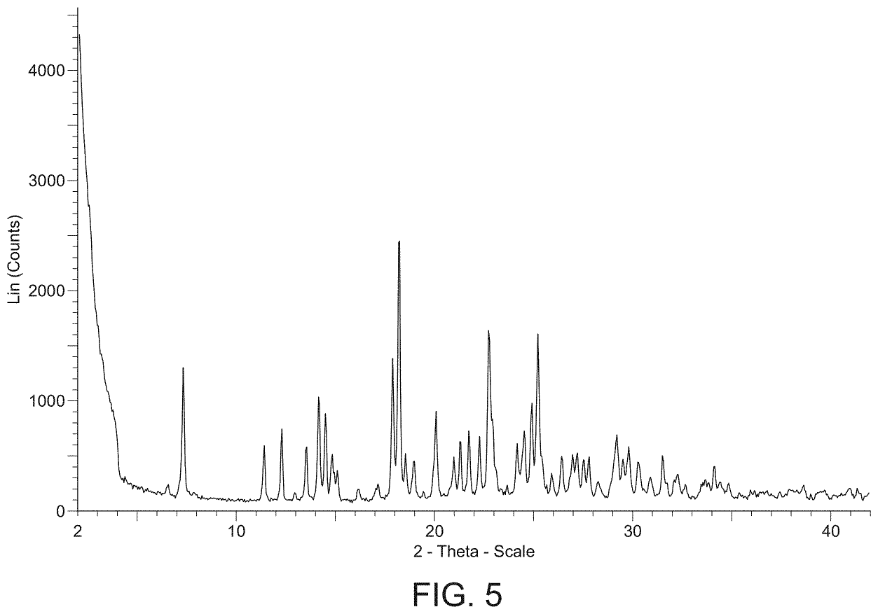

The molecular complex may have an X-ray powder diffraction pattern comprising one or more peaks (for example 1, 2, 3, 4, 5, 6, 7, 8, 9, 10, or 11 peaks) selected from the group consisting of about 6.5, 7.3, 7.8, 11.4, 12.3, 12.9, 13.6, 14.2, 14.5, 14.8, 15.1, 16.2, 17.1, 17.9, 18.2, 18.6, 19.0, 19.5, 20.1, 21.0, 21.3, 21.8, 22.3, 22.7, 23.7, 24.2, 24.5, 24.9, 25.2, 25.9, 26.4, 27.0, 27.2, 27.6, 27.8, 28.3, 29.2, 29.5, 29.8, 30.3, and 30.9 degrees two-theta ±0.2 degrees two-theta. In one embodiment, the molecular complex may have an X-ray powder diffraction pattern comprising peaks at about 7.3, 11.4, 12.3, 13.6, 14.2, 14.5, 17.9, 18.2, 20.1, 21.8, and 24.9 degrees two-theta ±0.2 degrees two-theta. In one embodiment, the molecular complex may have the X-ray powder diffraction pattern substantially as shown in FIG. 5. Without wishing to be bound by theory, the ratio of binimetinib to citric acid appears to be about 1 molecule of binimetinib:about 0.5 to about 2 molecules of citric acid, such as about 1 molecule of binimetinib:about 1 molecule of citric acid.

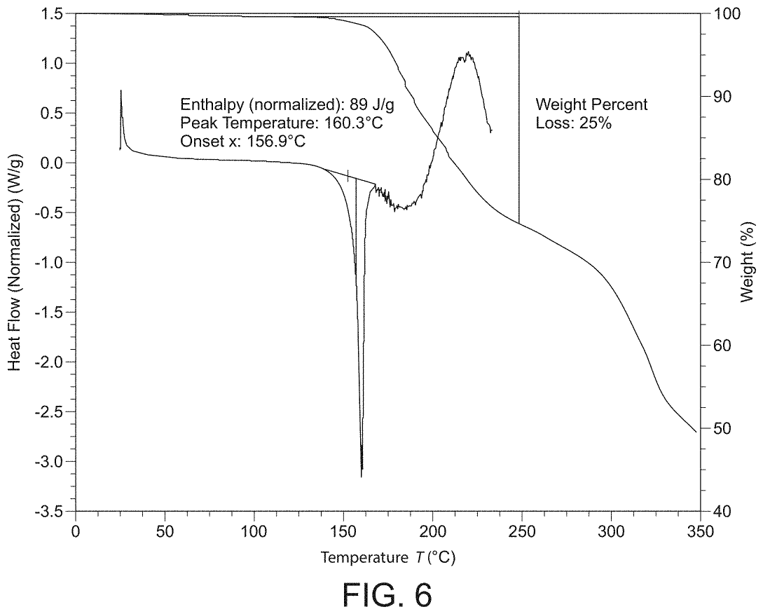

The molecular complex may have a DSC thermogram comprising an endothermal event with an onset temperature of about 156.9° C. The molecular complex may have a DSC thermogram comprising an endothermal event with a peak at about 160.3° C. In one embodiment, the molecular complex may have a DSC thermogram substantially as shown in FIG. 6.

The molecular complex may have a TGA thermogram comprising a mass loss of about 25% when heated from about 100° C. to about 250° C. In one embodiment, the molecular complex may have a TGA plot substantially as shown in FIG. 6.

The molecular complex of binimetinib citric acid may be prepared by a process comprising reacting binimetinib and citric acid using low energy ball milling or low energy grinding.

When low energy ball milling is utilised, the milling process may be controlled by various parameters including the speed at which the milling takes place, the length of milling time and/or the level to which the milling container is filled.

The speed at which the milling takes place may be from about 200 rpm to about 5000 rpm. In one embodiment, the speed may be from about 75 rpm to about 750 rpm. In another embodiment, the speed may be from about 80 rpm to about 600 rpm. In one embodiment, the speed may be about 500 rpm.

Low energy grinding may involve shaking the materials within a grinding container. In this instance, the grinding occurs via the impact and friction of the materials within the container. The process may be controlled by various parameters including the frequency at which the grinding takes place, the length of grinding time and/or the level to which the container is filled.

The frequency at which the grinding takes place may be from about 1 Hz to about 100 Hz. In one embodiment, the frequency may be from about 10 Hz to about 70 Hz. In another embodiment, the frequency may be from about 20 Hz to about 50 Hz. In one embodiment, the frequency may be about 30 Hz.

Milling or grinding media may be used to assist the reaction. In this instance, the incorporation of hard, non-contaminating media can additionally assist in the breakdown of particles where agglomeration has occurred, for example, as a result of the manufacturing process or during transit. Such breakdown of the agglomerates further enhances the reaction of binimetinib with citric acid. The use of milling/grinding media is well-known within the field of powder processing and materials such as stabilised zirconia and other ceramics are suitable provided they are sufficiently hard or ball bearings e.g. stainless steel ball bearings.

Alternatively, low energy grinding may comprise hand grinding with a pestle and mortar.

Regardless of whether milling or grinding is used, an improvement in the process can be made by controlling the particle ratio, the size of the milling/grinding media and other parameters as are familiar to the skilled person.

The length of milling or grinding time may be from about 1 minute to about 2 days, for example, about 10 minutes to about 5 hours, such as about 20 minutes to 3 hours. The length of milling or grinding time may be for a continuous or aggregate period of time. “Continuous” and “aggregate” are defined below.

The process may be carried out in a wet environment. For example, an alcohol solvent, such as methanol and/or ethanol, may added to the mixture of binimetinib and citric acid. The alcohol solvent (e.g. methanol and/or ethanol) can act to minimise particle welding. The addition of the alcohol solvent (e.g. methanol and/or ethanol) may be particularly helpful if the binimetinib and/or citric acid being reacted has agglomerated prior to use, in which case the alcohol solvent (e.g. methanol and/or ethanol) can assist with breaking down the agglomerates.

The quantity of solvent added is not particularly limiting provided sufficient solvent is added to moisten (i.e. “wet”) the admixture but not so large a quantity that the admixture becomes too liquid. The w/v ratio of total solids (binimetinib and citric acid) to total solvent added may be in the range of about 1 g total solids:about 0.1 to about 2 ml of total solvent added, such as about 1 g total solids:about 0.5 ml to about 1.5 ml of total solvent, e.g. about 1 g total solids:about 0.75 ml to about 1.25 ml of total solvent. In one embodiment, the w/v ratio of total solids (binimetinib and citric acid) to total solvent may be about 1 g total solids:about 1 ml of total solvent. The solvent may be added in one portion or more than one portions (e.g. 1, 2, 3, 4, or 5 portions).

The binimetinib may be present as the free base, anhydrate or as a solvate, such as binimetinib DMSO solvate.

The citric acid may be present as the free acid, anhydrate or hydrate, e.g. the monohydrate.

The citric acid may be present in stoichiometric or excess molar equivalents to the binimetinib. In one embodiment, the citric acid is present in stoichiometric quantities.

Alternatively, binimetinib citric acid molecular complex may be prepared by a process comprising the step of applying dual asymmetric centrifugal forces to a mixture of binimetinib and citric acid to form the molecular complex.

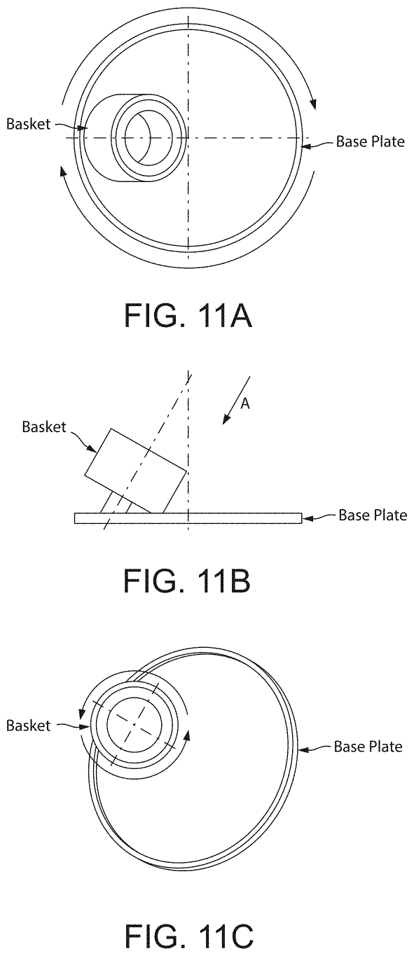

The molecular complex of binimetinib citric acid is formed using dual asymmetric centrifugal forces. By “dual asymmetric centrifugal forces” we mean that two centrifugal forces, at an angle to each other, are simultaneously applied to the particles. In order to create an efficient mixing environment, the centrifugal forces preferably rotate in opposite directions. The Speedmixer™ by Hauschild (http://www.speedmixer.co.uk/index.php) utilises this dual rotation method whereby the motor of the Speedmixer™ rotates the base plate of the mixing unit in a clockwise direction (see FIG. 11A) and the basket is spun in an anti-clockwise direction (see FIGS. 11B and 11C).

The process may be controlled by various parameters including the rotation speed at which the process takes place, the length of processing time, the level to which the mixing container is filled, the use of milling media and/or the control of the temperature of the components within the milling pot.

The dual asymmetric centrifugal forces may be applied for a continuous period of time. By “continuous” we mean a period of time without interruption. The period of time may be from about 1 second to about 10 minutes, such as about 5 seconds to about 5 minutes, for example, about 10 seconds to about 200 seconds e.g. 2 minutes.

Alternatively, the dual asymmetric centrifugal forces may be applied for an aggregate period of time. By “aggregate” we mean the sum or total of more than one periods of time (e.g. 2, 3, 4, 5 or more times). The advantage of applying the centrifugal forces in a stepwise manner is that excessive heating of the particles can be avoided. The dual asymmetric centrifugal forces may be applied for an aggregate period of about 1 second to about 20 minutes, for example about 30 seconds to about 15 minutes and such as about 10 seconds to about 10 minutes e.g. 6 minutes. In one embodiment, the dual asymmetric centrifugal forces are applied in a stepwise manner with periods of cooling therebetween. In another embodiment, the dual asymmetric centrifugal forces may be applied in a stepwise manner at one or more different speeds.

The speed of the dual asymmetric centrifugal forces may be from about 200 rpm to about 4000 rpm. In one embodiment, the speed may be from about 300 rpm to about 3750 rpm, for example about 500 rpm to about 3500 rpm. In one embodiment, the speed may be about 3500 rpm. In another embodiment, the speed may be about 2300 rpm.

The level to which the mixing container is filled is determined by various factors which will be apparent to the skilled person. These factors include the apparent density of the binimetinib and citric acid, the volume of the mixing container and the weight restrictions imposed on the mixer itself.

Milling media as described above may be used to assist the reaction. In certain embodiments, the dual asymmetric centrifugal forces may be applied in a stepwise manner in which milling media may be used for some, but not all, periods of time.

The process may be carried out in a wet environment. For example, an alcohol solvent, such as methanol and/or ethanol, may added to the mixture of binimetinib and citric acid. The alcohol solvent (e.g. methanol and/or ethanol) can act to minimise particle welding. The addition of the alcohol solvent (e.g. methanol and/or ethanol) may be particularly helpful if the binimetinib and/or citric acid being reacted has agglomerated prior to use, in which case the alcohol solvent (e.g. methanol and/or ethanol) can assist with breaking down the agglomerates.

The quantity of solvent added is not particularly limiting provided sufficient solvent is added to moisten (i.e. “wet”) the admixture but not so large a quantity that the admixture becomes too liquid. The w/v ratio of total solids (binimetinib and citric acid) to total solvent added may be in the range of about 1 g total solids:about 0.1 to about 2 ml of total solvent added, such as about 1 g total solids:about 0.5 ml to about 1.5 ml of total solvent, e.g. about 1 g total solids:about 0.75 ml to about 1.25 ml of total solvent.

In one embodiment, the w/v ratio of total solids (binimetinib and citric acid) to total solvent may be about 1 g total solids:about 1 ml of total solvent. The solvent may be added in one portion or more than one portions (e.g. 1, 2, 3, 4, or 5 portions).

When the dual asymmetric centrifugal forces are applied for an aggregate period of time, the wet or dry environment may be changed for each period of time. For example, the process may comprise a first period of time in which the environment is dry (i.e. binimetinib and citric acid are reacted together optionally with milling media in the absence of solvent), and a second period of time in which the environment is wet after the addition of solvent.

The binimetinib may be present as the free base, anhydrate or as a solvate, such as binimetinib DMSO solvate.

The citric acid may be present as the free acid, anhydrate or hydrate, e.g. the monohydrate.

The citric acid may be present in stoichiometric or excess molar equivalents to the binimetinib. In one embodiment, the citric acid is present in stoichiometric quantities.

Alternatively, binimetinib citric acid molecular complex may be prepared by a process comprising the steps of:
(a) providing an admixture of binimetinib and citric acid; and
(b) feeding the admixture through an extruder to form a binimetinib citric acid molecular complex.

The admixture is a blend of binimetinib and citric acid. The admixture may be prepared by mixing binimetinib and citric by any suitable means, e.g. by using a tubular blender, for a suitable period of time e.g. about 30 minutes. It is desirable but not essential to prepare a homogeneous blend of binimetinib and citric acid.

The binimetinib may be present as the free base, anhydrate or as a solvate, such as binimetinib DMSO solvate.

The citric acid may be present as the free acid, anhydrate or hydrate, e.g. the monohydrate.

The citric acid may be present in stoichiometric or excess molar equivalents to the binimetinib. In one embodiment, the citric acid is present in stoichiometric quantities.

The molecular complex does not form on preparing the admixture. The binimetinib and citric acid co-crystallise to form the molecular complex on feeding the admixture through the extruder.

An extruder typically includes a rotating screw or screws within a stationary barrel with a die located at one end of the barrel. Along the entire length of the screw, the co-crystallisation of the admixture is provided by the rotation of the screw(s) within the barrel. The extruder can be divided into at least three sections: a feeding section; a heating section and a metering section. In the feeding section, the admixture is fed into the extruder. The admixture can be directly added to the feeding section with or without the need of a solvent. In the heating section, the admixture is heated to a temperature such that the binimetinib and citric acid co-crystallise to form the molecular complex as the admixture transverses the section. A solvent may be optionally added in the heating section. After the heating section is an optional metering section in which the molecular complex may be extruded through a die into a particular shape, e.g., granules. The extruder may be a single screw extruder, a twin screw extruder, a multi screw extruder or an intermeshing screw extruder. In one embodiment, the extruder is a twin screw extruder e.g. a co-rotating twin screw extruder.

The admixture may be fed into the feeding section at any suitable speed. For example, the speed of the feeding section may be from about 1 rpm to about 100 rpm. In one embodiment, the speed may be from about 5 rpm to about 80 rpm. In one embodiment, the speed may be about 10 rpm. In another embodiment, the speed may be about 20 rpm.

In certain embodiments, solvent is added to the admixture as the admixture is fed into the feeding section. Alternatively or in addition, a solvent may be added one or more times (e.g. 1, 2, 3, 4, or 5 times) in one or more zones (e.g. 1, 2, 3, 4, or 5 zones) of the heating section as the admixture traverses the heating section. This may be advantageous in preventing the admixture drying out as the material moves through the heating section. The solvent may be an alcohol solvent, such as methanol and/or ethanol. In one embodiment, the alcohol solvent is methanol.

The quantity of solvent added is not particularly limiting provided sufficient solvent is added to moisten (i.e. “wet”) the admixture but not so large a quantity that the admixture becomes too liquid. When the extruder is a twin screw extruder, the w/v ratio of total solids (binimetinib and citric acid) to total solvent added may be in the range of about 1 g total solids:about 0.1 to about 2 ml of total solvent added, such as about 1 g total solids:about 0.5 ml to about 1.5 ml of total solvent, e.g. about 1 g total solids:about 0.75 ml to about 1.25 ml of total solvent. In one embodiment, the w/v ratio of total solids (binimetinib and citric acid) to total solvent is about 1 g total solids:about 1 ml of total solvent.

The heating section may be heated to a single temperature across its length or it may be divided into more than one (e.g. 2, 3, 4, or 5) zones, each of which may be heated independently of the other zones. The temperature of the heating section or each zone is not particularly limiting provided that on exiting the heating section the binimetinib and citric acid have co-crystallised to form the molecular complex and none of binimetinib, citric acid and/or the molecular complex have substantially degraded or substantially decomposed.

When the extruder is a twin screw extruder, the heating section may be divided into more than one zone as described above, and each zone may be independently heated to a temperature in the range of about ambient temperature (e.g. about 25° C.) to about 115° C.

When the extruder comprises screws, the screw (or screws) and the heating section may coincide i.e. the screw (or screws) may also be the heating section.

The speed at which the screw (or screws) rotate may be any suitable speed. For example, the speed of the screw (or screws) may be from about 1 rpm to about 500 rpm. In one embodiment, the speed may be from about 5 rpm to about 400 rpm, such as about 10 rpm to about 100 rpm. In one embodiment, the speed may be about 25 rpm. In another embodiment, the speed may be about 50 rpm. In another embodiment, the speed may be about 75 rpm.

The binimetinib citric acid molecular complex is recovered as a crystalline solid regardless of the process by which it is prepared. The crystalline molecular complex may be recovered by directly by filtering, decanting, centrifuging, or collecting the crystalline product. If desired, a proportion of the solvent (if present) may be evaporated prior to recovery of the crystalline solid.

Howsoever the crystalline molecular complex is recovered, the separated molecular complex may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

In another aspect, the present invention relates to a pharmaceutical composition comprising binimetinib citric acid molecular complex as described herein and a pharmaceutically acceptable excipient.

In another aspect, the present invention relates to a method for inhibiting MEK activity in a patient comprising administering a therapeutically effective amount of binimetinib citric acid molecular complex as described herein to the patient.

In another aspect, the present invention relates to a method for the treatment of a hyperproliferative disorder in a patient comprising administering a therapeutically effective amount of binimetinib citric acid molecular complex to the patient.

In another aspect, the present invention relates to binimetinib citric acid molecular complex as described herein for use in inhibiting MEK activity.

In another aspect, the present invention relates to binimetinib citric acid molecular complex as described herein for use in the treatment of a hyperproliferative disorder.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

General

XRPD Method

XRPD diffractograms were collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard collection method are:
Angular range: 2 to 42° 2θ
Step size: 0.05° 2θ
Collection time: 0.5 s/step (total collection time: 6.40 min)

DSC Method

DSC data were collected on a TA Instruments Q2000 or Discovery TGA equipped with a 50 position auto-sampler. Typically, 0.5-3 mg of each sample, in a pin-holed aluminium pan, was heated at 10° C./min from 25° C. to 300° C. (for binimetinib DMSO solvate) or 10° C./min from 25° C. to 235° C. (for binimetinib citric acid molecular complex). A purge of dry nitrogen at 50 ml/min was maintained over the sample.

The instrument control software was TRIOS and the data were analysed using TRIOS or Universal Analysis.

TGA Method

TGA data were collected on a TA Instruments Q500 or Discovery TGA, equipped with a 16 position auto-sampler. Typically, 5-10 mg of each sample was loaded onto a pre-tared aluminium DSC pan and heated at 10° C./min from ambient temperature to 350° C. A nitrogen purge at 60 ml/min was maintained over the sample.

The instrument control software was Advantage for Q Series and Thermal Advantage and the data were analysed using TRIOS or Universal Analysis.

Solution State NMR

¹H NMR spectra were collected on a Bruker 400 MHz instrument equipped with an auto-sampler and controlled by a DRX400 console. Samples were prepared in MeOH-d₄ solvent (binimetinib DMSO solvate) or DMSO-d₆ solvent (binimetinib citric acid molecular complex), unless otherwise stated. Automated experiments were acquired using ICON-NMR configuration within Topspin software, using standard Bruker-loaded experiments (¹H). Off-line analysis was performed using ACD Spectrus Processor.

FT-IR Method

Data were collected on a Perkin-Elmer Spectrum One fitted with a universal Attenuated Total Reflectance (ATR) sampling accessory from 4000-650 cm⁻¹ over 16 scans. The data were collected using Spectrum software and processed using ACD Spectrus Processor.

Raman Method

Data were collected on a Renishaw inVia Qontor. Instrument control and background subtraction processing were completed using WiRE. Data presentation was completed using ACD Spectrus Processor.

Method: excitation source, λₑₓ=785 nm laser; Raman shift range: 150-1900 cm⁻¹; Exposure time: 30 s; Accumulations: 3

Single Crystal X-Ray Diffraction (SCXRD)

Data were collected on a Rigaku Oxford Diffraction Supernova Dual Source, Cu at Zero, Atlas CCD diffractometer equipped with an Oxford Cryosystems Cobra cooling device. The data were collected using Cu Kα radiation as stated in the experimental tables. Structures were solved and refined using the Bruker AXS SHELXTL suite or the OLEX² crystallographic software. Full details can be found in the CIF. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter. A reference diffractogram for the crystal structure was generated using Mercury.

Chemical Purity Determination by HPLC

Purity analysis was performed on an Agilent HP1100/Infinity II 1260 series system equipped with a diode array detector and using ChemStation or OpenLAB software. The full method details are provided below:

TABLE 1
HPLC method for chemical purity determinations

| Parameter | Value | | |
|---|---|---|---|
| Type of method | Reverse phase with gradient elution | | |
| Sample Preparation | 0.5 mg/ml in acetonitrile:water 1:1 | | |
| Column | Supelco Ascentis Express C18, | | |
| | 100 × 4.6 mm, 2.7 μm | | |
| Column Temperature (° C.) | 25 | | |
| Injection (μl) | 5 | | |
| Wavelength, Bandwidth (nm) | 255, 90 | | |
| Flow Rate (ml/min) | 2 | | |
| Phase A | 0.1% TFA in water | | |
| Phase B | 0.085% TFA in acetonitrile | | |
| Timetable | Time (min) | % Phase A | % Phase B |
| | 0 | 95 | 5 |
| | 6 | 5 | 95 |
| | 6.2 | 95 | 5 |
| | 8 | 95 | 5 |

### Abbreviations

DMSO dimethylsulfoxide
eq. equivalent
HME hot melt extrusion
IPA isopropanol
MeOH methanol
min minute

### Example 1—Binimetinib DMSO Solvate

Increasing aliquots of DMSO were added to binimetinib (15 mg, 97.5% pure) at ambient temperature until dissolution was observed (total of 300 μl). Between additions the sample was shaken at ambient temperature for ca. 30 seconds. The solvent was evaporated at ambient conditions.

### Example 2—Binimetinib DMSO Solvate

Binimetinib (ca. 50 mg, 97.5% pure) was suspended in DMSO (total 20 volumes; 1 ml in total) and stirred at ambient temperature for 15 minutes to give a clear solution. Increasing aliquots of the selected anti-solvent (IPA or water) were added (total of 60 volumes; 3 ml in total) and the samples were stirred at ambient temperature. After stirring overnight, aliquots of the suspensions were filtered and dried under suction for a few minutes prior to XRPD analysis. The bulk samples were filtered and dried under vacuum for ca. 1 hour.

### Example 3—Binimetinib DMSO Solvate

Binimetinib (ca. 2 g, 97.5% pure) was treated with DMSO (15 volumes; 30 ml in total) and stirred at 70° C. for 15 minutes. The solution was polish filtered and stirred at 70° C. for 5 minutes then cooled to 25° C. at 1° C./min. The clear solution was treated with IPA (60 volumes; 120 ml in total) and stirred at 25° C. After 4 hours, a suspension was obtained. An aliquot was filtered and dried under suction for few minutes prior to characterisation.

The bulk sample was filtered and dried under suction for 20 minutes and dried in vacuum oven at 25° C. for 4 hours.

### Example 4—Binimetinib DMSO Solvate

Binimetinib (ca. 8 g, 97.5% pure) was suspended in DMSO (1 vol; 8 ml) and stirred at 100° C. for 70 minutes. As the sample remained a suspension, additional DMSO (0.5 vol; 4 ml) was added. After 25 minutes, a clear solution was obtained.

The solution was cooled to 25° C. at 1° C./min. IPA (9 vol; 72 ml) was added over 25 minutes to the resulting suspension. The sample was stirred at 25° C. for 80 minutes then cooled to 5° C. at 1° C./min and stirred at 5° C. for ca. 36 hours. The sample was filtered and washed twice with 1 vol of IPA (16 ml in total). The sample was dried under suction for <1 minute and dried under vacuum for 1 hour at ambient temperature. Yield:80%

### Example 5—Characterisation of Binimetinib DMSO Solvate

The crystal structure of binimetinib DMSO solvate was determined at 100 K and a summary of the structural data can be found in Tables 1 and 2. The binimetinib DMSO solvate crystallises in the orthorhombic system, space group P2₁2₁2₁ with the final R1 [I>2σ(I)]=3.06. The structure was identified as depicted in FIG. 1 and the asymmetric unit found to contain one fully ordered molecule of Binibmetinib and one fully ordered molecule of DMSO as depicted in FIG. 2.

TABLE 1
Crystal data for binimetinib DMSO solvate

| Crystallisation solvents | DMSO | |
|---|---|---|
| Crystallisation method | Slow evaporation | |
| Empirical formula | C₁₉H₂₁BrF₂N₄O₄S | |
| Formula weight | 519.37 | |
| Temperature | 100(2) K | |
| Wavelength | 1.54184 Å | |
| Crystal size | 0.450 × 0.080 × 0.020 mm | |
| Crystal habit | colourless lath | |
| Crystal system | Orthorhombic | |
| Space group | P2₁2₁2₁ | |
| Unit cell dimensions | a = 4.6450(2) Å | α = 90° |
| | b = 20.5765(7) Å | β = 90° |
| | c = 22.4492(5) Å | γ = 90° |

| | |
|---|---|
| Volume | 2145.65(13) Å³ |
| Z | 4 |
| Density (calculated) | 1.608 Mg/m³ |
| Absorption coefficient | 3.989 mm⁻¹ |
| F(000) | 1056 |

TABLE 2
Data collection and structure refinement for binimetinib DMSO solvate

| | |
|---|---|
| Diffractometer | SuperNova, Dual, Cu at zero, Atlas |
| Radiation source | SuperNova (Cu) X-ray Source, CuKa |
| Data collection method | omega scans |
| Theta range for data collection | 3.938 to 70.530° |
| Index ranges | −5 ≤ h ≤ 5, −25 ≤ k ≤ 25, −27 ≤ l ≤ 24 |
| Reflections collected | 41791 |
| Independent reflections | 4091 [R(int) = 0.0763] |
| Coverage of independent | 100.0% |
| reflections | |
| Variation in check reflections | n/a |
| Absorption correction | Semi-empirical from equivalents |
| Max. and min. transmission | 1.00000 and 0.49384 |
| Structure solution technique | Direct Methods |
| Structure solution program | SHELXTL (Sheldrick, 2013) |
| Refinement technique | Full-matrix least-squares on F² |
| Refinement program | SHELXL-2013 (Sheldrick, 2013) |
| Function minimized | Σw(Fₒ² − F_{c}²)² |
| Data/restraints/parameters | 4091/0/295 |
| Goodness-of-fit on F² | 1.022 |
| Δ/σₘₐₓ | 0.001 |
| Final R indices | R1 = 0.0306, wR2 = 0.0764 |
| 3900 data; I > 2σ(I) | R1 = 0.0328, wR2 = 0.0784 |
| all data | |
| Weighting scheme | w = 1/[σ²(Fₒ²) + (0.0438P)² + 1.2905P] |
| | where P = (Fₒ² − 2F_{c}²)²/3 |
| Absolute structure parameter | −0.041(11) |
| Extinction coefficient | n/a |
| Largest diff. peak and hole | 0.332 and −0.281 eÅ⁻³ |

Table 3 provides an XRPD peak listing for binimetinib DMSO solvate.

TABLE 3

| Angle | Intensity |
|---|---|
| (2-Theta °) | (%) |
| 5.8 | 55.9 |
| 7.9 | 13.4 |
| 8.9 | 53.5 |
| 12.5 | 8.2 |
| 13.4 | 13.1 |
| 14.5 | 45.9 |
| 15.1 | 7.0 |
| 17.1 | 11.8 |
| 17.6 | 100.0 |
| 17.9 | 5.9 |
| 18.8 | 59.3 |
| 19.7 | 11.2 |
| 20.1 | 37.6 |
| 20.3 | 21.2 |
| 21.0 | 26.1 |
| 21.8 | 22.9 |
| 22.2 | 10.1 |
| 22.7 | 14.6 |
| 22.8 | 17.2 |
| 23.3 | 20.5 |
| 23.5 | 28.5 |
| 24.2 | 13.3 |
| 24.5 | 5.2 |
| 25.2 | 13.0 |
| 25.8 | 43.9 |
| 26.1 | 25.3 |
| 26.8 | 10.7 |
| 27.0 | 9.8 |
| 27.7 | 9.8 |
| 27.8 | 14.1 |
| 28.4 | 10.6 |
| 28.7 | 11.6 |
| 29.0 | 14.0 |
| 29.2 | 8.6 |
| 29.8 | 3.8 |
| 30.1 | 6.5 |
| 30.3 | 6.0 |
| 30.7 | 4.8 |

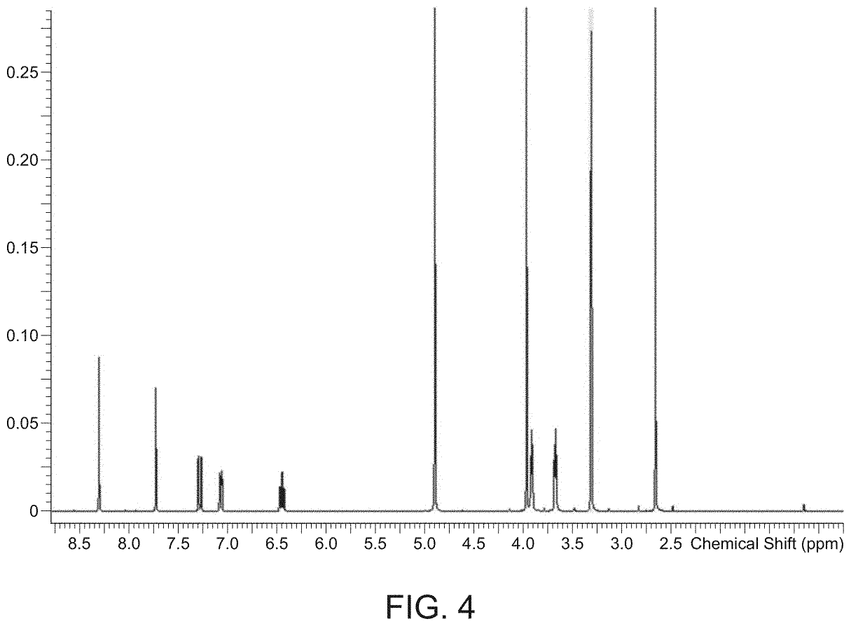

Binimetinib DMSO solvate was also characterised as follows:
TGA and DSC analysis (see FIG. 3); and
¹H-NMR spectroscopy (see FIG. 4).

### Example 6—Binimetinib Citric Acid Molecular Complex

Binimetinib (ca. 30 mg) and 1.0 eq. (ca. 13 mg) of citric acid were dispensed into an HPLC vial and two stainless steel grinding balls (3 mm diameter) added. Solvent was added (MeOH, 10 μl) and the sample was subjected to grinding on a Fritsch planetary mill (500 rpm, 2 hour duration). The solid obtained was analysed by XRPD and was identified as binimetinib citric acid molecular complex.

### Example 7—Binimetinib Citric Acid Molecular Complex

Binimetinib (500 mg) and 1.0 eq. of citric acid (ca. 218 mg) were dispensed into a grinding jar (25 ml) with one zirconia grinding ball (20 mm diameter) added. Methanol was added (90 μl) and the sample was subjected to grinding on a Retsch mill (30 Hz, 30 minutes). The solid obtained was analysed by XRPD and was identified as binimetinib citric acid molecular complex.

### Example 8—Binimetinib Citric Acid Molecular Complex

Binimetinib DMSO solvate (60 mg,) and 2.0 eq. of citric acid (ca. 44 mg) were dispensed into an HPLC vial and two stainless steel grinding balls (3 mm diameter) added. MeOH was added (30 μl) and the sample was subjected to grinding on a Fritsch planetary mill (500 rpm, 20 minutes). The solid obtained post grinding was analysed by XRPD and was identified as binimetinib citric acid molecular complex.

### Example 9—Binimetinib Citric Acid Molecular Complex

Binimetinib (699 mg) and 1.0 eq. of citric acid (305 mg) were added to a plastic container (PP10) and mixed at 3500 rpm for 2 minutes on a DAC150-FV2-K mixer from Speedmixer™. To the mixture ten ball bearings (3 mm diameter) were added with MeOH (235 μl) and mixed at 2300 rpm for 2 minutes. The ball bearings were removed and the sample re-mixed for 1 minute at 3500 rpm yielding a mixture of agglomerates. The ball bearings were added again and milled at 3500 rpm for 1 minute to yield powder and agglomerates of powder. The solid obtained was analysed by XRPD and was identified as binimetinib citric acid molecular complex.

### Example 10—Characterisation of Binimetinib Citric Acid Molecular Complex

Table 4 provides an XRPD peak listing for binimetinib citric acid molecular complex.

TABLE 4

| Angle | Intensity |
|---|---|
| (2-Theta °) | (%) |
| 6.5 | 9.5 |
| 7.3 | 52.9 |
| 7.8 | 6.5 |
| 11.4 | 23.9 |
| 12.3 | 30.2 |
| 12.9 | 6.4 |
| 13.6 | 23.5 |
| 14.2 | 42.0 |
| 14.5 | 35.8 |
| 14.8 | 20.7 |
| 15.1 | 14.7 |
| 16.2 | 7.7 |
| 17.1 | 9.7 |
| 17.9 | 56.4 |
| 18.2 | 100.0 |
| 18.6 | 21.0 |
| 19.0 | 18.1 |
| 19.5 | 6.9 |
| 20.1 | 36.7 |
| 21.0 | 19.7 |
| 21.3 | 25.4 |
| 21.8 | 29.4 |
| 22.3 | 27.3 |
| 22.7 | 66.7 |
| 23.7 | 9.2 |
| 24.2 | 24.7 |
| 24.5 | 29.4 |
| 24.9 | 39.7 |
| 25.2 | 65.5 |
| 25.9 | 13.6 |
| 26.4 | 19.9 |
| 27.0 | 20.6 |
| 27.2 | 21.2 |
| 27.6 | 18.7 |
| 27.8 | 19.8 |
| 28.3 | 10.6 |
| 29.2 | 27.9 |
| 29.5 | 18.9 |
| 29.8 | 23.5 |
| 30.3 | 17.8 |
| 30.9 | 12.2 |

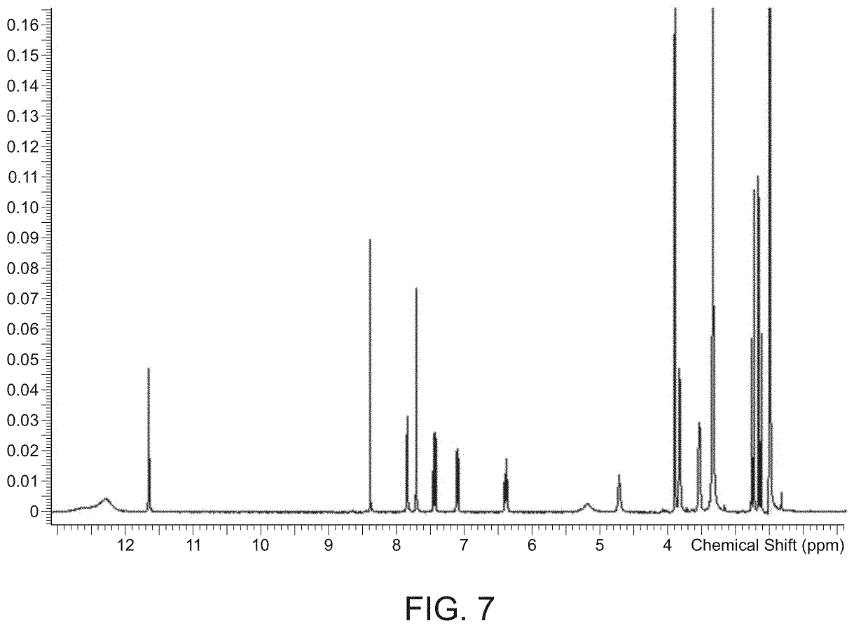

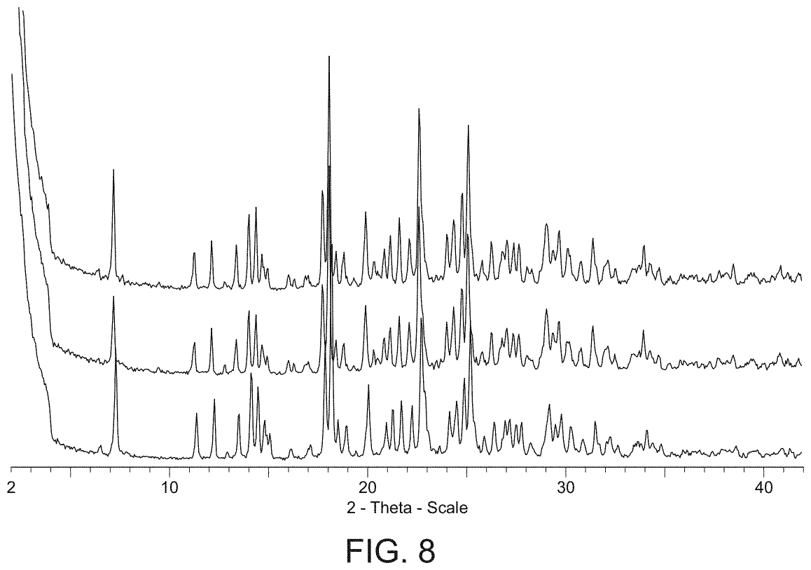

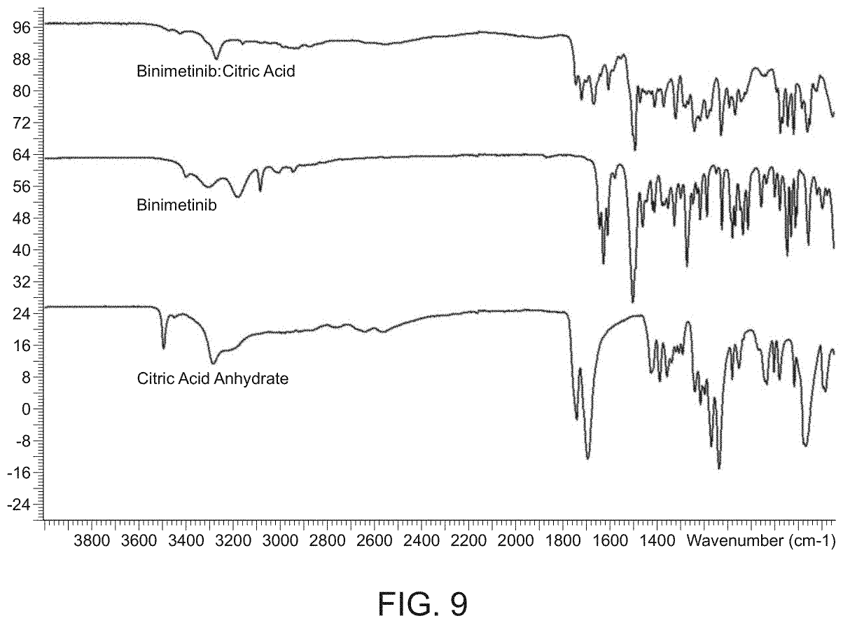

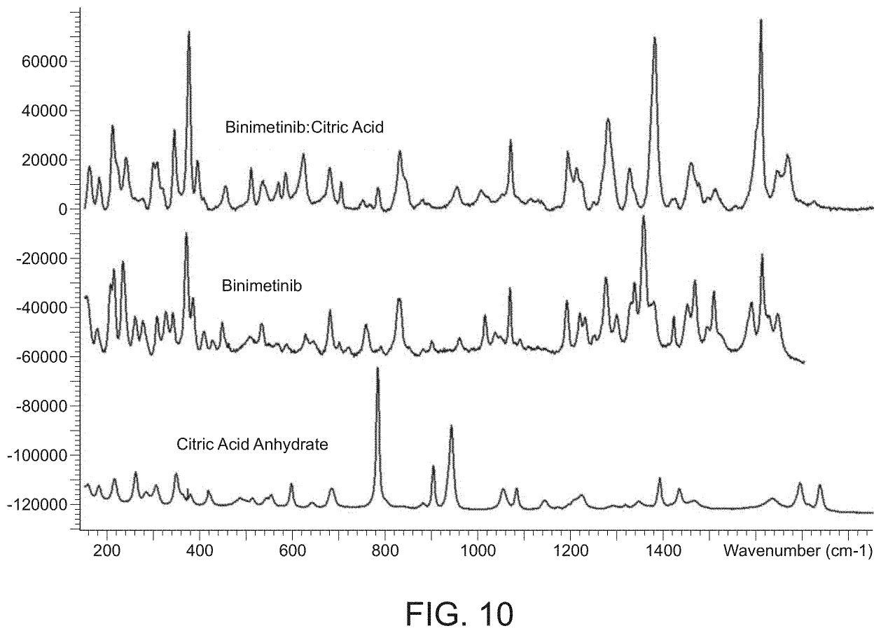

Binimetinib citric acid molecular complex was also characterised as follows:
TGA and DSC analysis (see FIG. 6); and
¹H-NMR spectroscopy (see FIG. 7).
Stability studies at three storage conditions. FIG. 8 shows an XRPD overlay of binimetinib citric acid before storage (bottom), after storage at 40° C./75% RH for 10 days (middle) and after storage at 25° C./97% RH after 10 days (top). The molecular complex remains stable under two different temperature and humidity conditions for at least 10 days.
FT-IR analysis (see FIG. 9). FIG. 9 shows an FT-IR overlay of (a) binimetinib citric acid molecular complex, (b) binimetinib free base, and (c) citric acid anhydrate.
Raman analysis (see FIG. 10). FIG. 10 shows a Raman overlay of (a) binimetinib citric acid molecular complex, (b) binimetinib free base, and (c) citric acid anhydrate.
Binimetinib citric acid was analysed by ¹H, ¹³C and ¹⁵N solid state MAS NMR, DFT and machine learning computational analysis. The data obtained (not shown):
   indicates that neither nitrogen in the methylimidazole part of the binimetinib molecular is protonated in binimetinib:citric acid. The binimetinib:citric acid sample therefore is consistent with being a co-crystal, rather than a salt.
   a hydrogen bonded carboxylic acid proton with a long O—H bond length (estimated around 1.1 Å) was observed in the binimetinib:citric acid sample.
   two crystallographically inequivalent binimetinib molecules are present in the binimetinib:citric acid unit cell.

### Pestle and Mortar Examples

To ensure homogeneous mixing, hand-grinding was used for a mixture of binimetinib and citric acid, wetted with either methanol or ethanol.

### Example 11—Binimetinib Citric Acid Molecular Complex

Binimetinib (4.0 g) and citric acid (1.74 g) were added into a large marble pestle and mixed with grinding by hand using a mortar, in the presence of methanol (2 ml), for 10 minutes. The components were then wetted with further methanol to 5.7 ml in total, and ground by hand for 10 minutes. After this time, the wet paste was allowed to air-dry for 10 minutes, then re-ground for 10 minutes. This process was repeated once more, until a dry solid was obtained, which adhered to the pestle. The solid was scraped off into a beaker and dried under vacuum at 50° C. overnight. The resulting solid was analysed by XRPD, 1H NMR and thermal techniques to confirm formation of the molecular complex.

### Example 12—Binimetinib Citric Acid Molecular Complex

Binimetinib (1.00 g) and citric acid monohydrate (0.48 g) were added into a large pestle and mixed with grinding by hand, in the presence of ethanol (1 vol, 1.5 ml) for 10 minutes. After this time, the wet paste was allowed to air-dry for 10 minutes, then re-ground for 10 minutes. This process was repeated once more, until a dry solid was obtained, which adhered to the pestle. The solid was scraped off into a 20 ml vial and briefly air-dried. The resulting solid was analysed by XRPD to confirm formation of the molecular complex.

Hot Melt Extrusion (HME) Examples

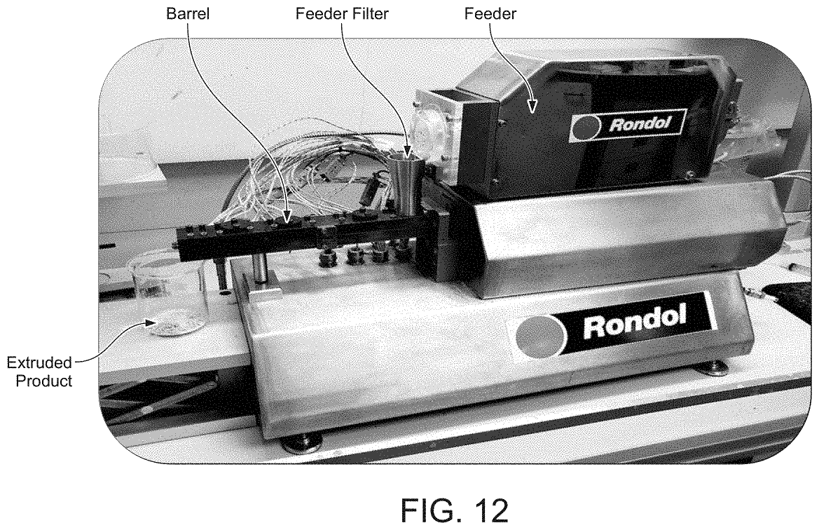

A Rondol Microlab 10 mm hot melt extruder (in this instance a twin screw extruder) (see FIG. 12) was used for all experiments. The components of the extruder to note are the Feeder, the Feeder Filter which supplies the mixed starting material to the extruder and the barrel which houses the co-rotating twin screws. The extruder barrel has four controllable temperature zones (excluding the die zone). For these experiments, the die was not used.

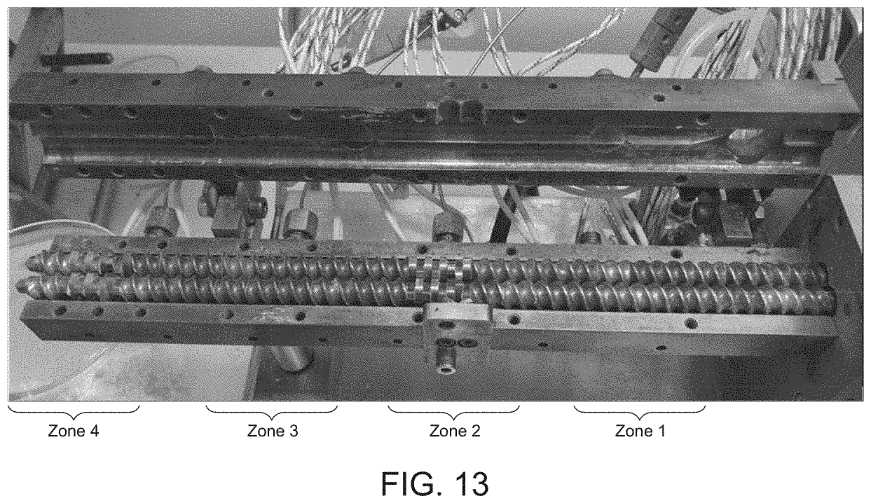

The temperature of the extruder barrel was varied between 25° C. to 115° C. Stoichiometric (molar) blends of the starting components were prepared and mixed using a Tubular blender for 30 minutes prior to being charged into the extruder. Feeder speed can be varied between 10 rpm and 80 rpm and the screw speed could be increased to a maximum of 400 rpm. The screw configuration is shown in FIG. 13. The screw design was set up with alternating 10 mm segments for conveying and mixing. Zone 1 is a purely conveying zone with minimal mixing capacity. Zone 2 is a high mixing element. This conveying and mixing element are repeated for Zone 3 and Zone 4, respectively. Each experiment processed between 3 g and 10 g of material.

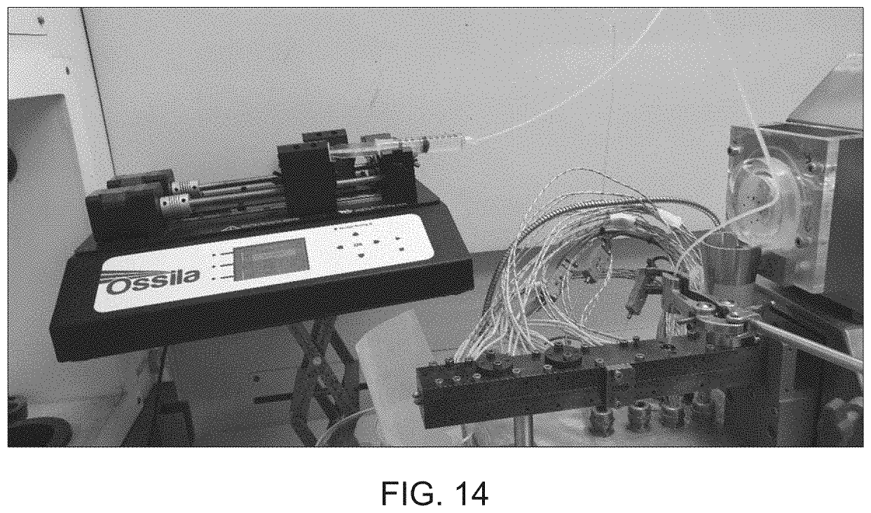

For the HME experiments involving solvent addition, a mechanical syringe pump was used to precisely control the rate of solvent addition. The solvent addition was performed in Zone 1. This is shown in FIG. 14.

HME Studies

HME Temperature Experiments

Procedure

Binimetinib (3.00 g) and citric acid monohydrate (1.42 g) were physically mixed to give a homogenous sample by blending on the tubular blender for 30 minutes. The mixtures were passed through the hot melt extruder (HME) at multiple temperatures.

Results and Discussion

Several HME experiments were performed using a binimetinib/citric acid monohydrate physical mixture (1:1) without the use of solvent. In these studies, the impact of temperature on the physical appearance and consistency of the material, as well as whether crystallisation of the molecular complex could be achieved using a solvent free procedure was investigated. In addition, HPLC analysis was performed to study the effect of temperature on chemical integrity (purity).

In these studies, the throughput (or feeding rate) was kept consistent at 20 rpm. However, it was found that the screw speed had a distinct impact of the consistency of the material passing through the instrument. At lower screw speed, the material was found to pass through the instrument without any changes in colour or physical appearance. However, at higher screw speed, the increased mechanical stress appeared to cause some degree of discolouration on the material.

With respect to temperature influence on material, the material was found to pass through the instrument when the barrel was kept at 25° C. However, XRPD analysis revealed no conversion to the desired molecular complex had occurred. Extrusion at 115° C. was also performed as at this temperature the citric acid monohydrate was expected to dehydrate, providing some water solvent which may facilitate crystallisation.

However, the material which exited to extruder was observed to be molten and grey in colour. XRPD analysis of this material was consistent with the binimetinib citric acid molecular complex. The HPLC showed significant degradation had occurred (89.1% purity reading).

These results are summarised in the Table 5 below:

TABLE 5
HME temperature experiments in the absence of solvent

| | Temperature | | | | | | | | |
|---|---|---|---|---|---|---|---|---|---|
| | Zone | Zone | Zone | Zone | Feeder | Screw | | | |
| Example | 1 | 2 | 3 | 4 | speed | speed | Observations | XRPD | HPLC |
| 13* | 25° C. | | | | 20 rpm | 25 rpm | | Starting | N/A |
| | | | | | | | | material | |
| 14 Sample 1 | 115° C. | | | | 20 rpm | 50 rpm | Sample 1 was | Binimetinib | 95.7% |
| | | | | | | | extracted from | citric acid | |
| | | | | | | | material in | molecular | |
| | | | | | | | Zone 1/Zone 2. | complex | |
| | | | | | | | Zone 1/Zone 2: | | |
| | | | | | | | material was pink | | |
| 14 Sample 2 | | | | | | | Sample 2 was | Binimetinib | 94.7% |
| | | | | | | | extracted from | citric acid | |
| | | | | | | | material in Zone 3. | molecular | |
| | | | | | | | Zone 3: material | complex | |
| | | | | | | | was dark pink | | |
| 14 Sample 3 | | | | | | | Sample 3 was | Binimetinib | 89.1% |
| | | | | | | | extracted from | citric acid | |
| | | | | | | | material in Zone 4. | molecular | |
| | | | | | | | Zone 4: material | complex^{#} | |
| | | | | | | | was molten and grey | | |

*comparative
^{#}poorly crystalline
N/A not applicable

Wet Extrusion with Methanol

Binimetinib (3.00 g) and citric acid monohydrate (1.42 g) were physically mixed to give a homogenous sample by blending on the tubular blender for 30 minutes. The mixtures were passed through an extruder with MeOH addition added dropwise to Zone 1.

Results and Discussion

The binimetinib and citric acid was added to the extruder feeder in a 1:1 ratio. A series of screening experiments were conducted in which the feeder speed was adjusted to 10-20 rpm and the screw speed was kept at 50 rpm. The screening parameters focused on the relationship between extruder temperature and rate of solvent addition. The solvent chosen for these experiments was MeOH.

Two experiments were performed at low temperature (25° C.) at two different solvent addition rates. Extrusion with a high rate of MeOH addition (10 μl/sec) resulted in the material exiting the barrel too wet. Decreasing the rate of MeOH to 2.5 μl/sec ensured the physical mixture remained in a good solid consistency. The extruded material from the 2.5 μl/sec MeOH addition (25° C.) was characterised by XRPD, and HPLC. The XRPD was consistent with binimetinib citric acid molecular complex.

Two additional screening experiments were performed in which the MeOH addition rate was increased to 5 μl/sec and the temperature was increased to 50° C. and 60° C., respectively. Characterisation of the extruded materials all confirmed generation of the binimetinib citric acid molecular complex. In addition, reduced clumping at the MeOH addition site was observed.

These results are summarised in the Table 6 below:

TABLE 6
HME temperature experiments in the presence of methanol

| | Temperature | | | | | | | | |
|---|---|---|---|---|---|---|---|---|---|
| | Zone | Zone | Zone | Zone | Feeder | Screw | | | |
| Example | 1 | 2 | 3 | 4 | speed | speed | Observations | XRPD | HPLC |
| 17* | 25° C. | | | | 20 rpm | 50 rpm | MeOH added to | N/A | N/A |
| | | | | | | | 10 μl/sec. Material | | |
| | | | | | | | coming out of | | |
| | | | | | | | extruder too wet. | | |
| 18 | 25° C. | | | | | | MeOH added at | Binimetinib | 96.3% |
| | | | | | | | 2.5 μl/sec. | citric acid | |
| | | | | | | | | molecular | |
| | | | | | | | | complex | |
| 19 | 50° C. | | | 60° C. | | | MeOH added at | Binimetinib | 98.4% |
| | | | | | | | 5 μl/sec. | citric acid | |
| | | | | | | | | molecular | |
| | | | | | | | | complex | |
| 20 | 60° C. | | | | 10 rpm | 50 rpm | MeOH added at | Binimetinib | 98.3% |
| | | | | | | | 5 μl/sec. | citric acid | |
| | | | | | | | | molecular | |
| | | | | | | | | complex | |

*comparative
N/A not applicable

1. A molecular complex of binimetinib which is crystalline binimetinib DMSO solvate.

2. The molecular complex according to claim 1 having an X-ray powder diffraction pattern comprising one or more peaks selected from the group consisting of about 5.8, 7.9, 8.9, 12.5, 13.4, 14.5, 15.1, 17.1, 17.6, 17.9, 18.8, 19.7, 20.1, 20.3, 21.0, 21.8, 22.2, 22.7, 22.8, 23.3, 23.5, 24.2, 24.5, 25.2, 25.8, 26.1, 26.8, 27.0, 27.7, 27.8, 28.4, 28.7, 29.0, 29.2, 29.8, 30.1, 30.3, and 30.7 degrees two-theta ±0.2 degrees two-theta.

3. The molecular complex according to claim 2 having an X-ray powder diffraction pattern comprising peaks at about 5.8, 8.9, 14.5, 17.6, 18.8, 20.1, 23.5, and 25.8 degrees two-theta ±0.2 degrees two-theta.

4. The molecular complex according to claim 1, which has a DSC thermogram comprising an endothermal event with a peak at about 133.9° C. and another endothermal event with a peak at about 221.3° C.

5. A process for preparing binimetinib DMSO solvate, the process comprising the steps of:
(a) contacting binimetinib with DMSO; and
(b) forming a solution of binimetinib in DMSO.

6. The process according to claim 5, further comprising the step of recovering binimetinib DMSO solvate as a crystalline solid.

7. A molecular complex which is a crystalline molecular complex of binimetinib and citric acid.

8. The molecular complex according to claim 7, which has an X-ray powder diffraction pattern comprising one or more peaks selected from the group consisting of about 6.5, 7.3, 7.8, 11.4, 12.3, 12.9, 13.6, 14.2, 14.5, 14.8, 15.1, 16.2, 17.1, 17.9, 18.2, 18.6, 19.0, 19.5, 20.1, 21.0, 21.3, 21.8, 22.3, 22.7, 23.7, 24.2, 24.5, 24.9, 25.2, 25.9, 26.4, 27.0, 27.2, 27.6, 27.8, 28.3, 29.2, 29.5, 29.8, 30.3, and 30.9 degrees two-theta ±0.2 degrees two-theta.

9. The molecular complex according to claim 8, which has an X-ray powder diffraction pattern comprising peaks at about 7.3, 11.4, 12.3, 13.6, 14.2, 14.5, 17.9, 18.2, 20.1, 21.8, and 24.9 degrees two-theta ±0.2 degrees two-theta.

10. The molecular complex according to claim 7, which has a DSC thermogram comprising an endothermal event with a peak at about 160.3° C.

11. A process for preparing the crystalline molecular complex of binimetinib and citric acid of claim 7, which process comprises using low energy ball milling or low energy grinding to form the crystalline molecular complex.

12. A process for preparing the crystalline molecular complex of binimetinib and citric acid of claim 7, which process comprises the step of applying dual asymmetric centrifugal forces to a mixture of binimetinib and citric acid to form the crystalline molecular complex.

13. A process for preparing the crystalline molecular complex of binimetinib and citric acid of claim 7, which process comprising the steps of:
(a) providing an admixture of binimetinib and citric acid; and
(b) feeding the admixture through an extruder to form a binimetinib citric acid molecular complex.

14. A pharmaceutical composition comprising the molecular complex according to claim 1 and a pharmaceutically acceptable excipient.

15. A pharmaceutical composition comprising the molecular complex according to claim 7 and a pharmaceutically acceptable excipient.

16. A method for inhibiting MEK activity in a patient comprising administering a therapeutically effective amount of the molecular complex of claim 1 to the patient.

17. A method for inhibiting MEK activity in a patient comprising administering a therapeutically effective amount of the molecular complex of claim 7 to the patient.

18. A method for the treatment of a hyperproliferative disorder in a patient comprising administering a therapeutically effective amount of the molecular complex of claim 1 to the patient.

19. A method for the treatment of a hyperproliferative disorder in a patient comprising administering a therapeutically effective amount of the molecular complex of claim 7 to the patient.
